(19) United States
(12) Patent Application Publication
(10) Pub. No.: US 2025/0000404 A1
(43) Pub. Date: Jan. 2, 2025

(54) Systems and Methods for Monitoring Plasma Overcollection

(71) Applicant: Terumo BCT, Inc., Lakewood, CO (US)

(72) Inventors: Marlene BAINBRIDGE, Lakewood, CO (US); Logan FENDER, Denver, CO (US)

(21) Appl. No.: 18/217,151

(22) Filed: Jun. 30, 2023

(51) Int. Cl. A61B 5/15; A61B 5/00

(52) U.S. Cl. CPC A61B 5/150946; A61B 5/150755; A61B 5/150801; A61B 5/746

(57) ABSTRACT

A method for using an automated blood collection system to collect blood components from a subject includes separating plasma from whole blood as received from the subject; initiating an alert when a predicted accumulated volume loss is greater than a configured removal level, the predicted accumulated volume loss depending on an accumulated volume that includes the separated volume of plasma and the configured removal level being defined by a hypovolemic level or the pre-selected maximum for the subject; and in response to the alert and feedback from an operator of the automated blood collection system, ending the collection process, continuing the collection, or initiating platelet and red blood cell separation.

### FIELD

The present disclosure relates to systems and methods for monitoring overcollection during blood component collection.

### BACKGROUND

This section provides background information related to the present disclosure which is not necessarily prior art.

Blood collection systems for collecting blood from healthy donors for later emergency and/or medical treatment and/or therapeutic uses generally fall into two broad categories: manual blood collection systems and automated blood collection systems. Manual blood collection systems are those commonly seen and used, for example, in community blood drives, where blood from healthy donors is collected by gravity flow into one or more blood collection containers and later separated (using, for example, centrifuge systems) into one or more components, such as red blood cells, plasma, and/or platelets, which are then used for the emergency and/or medical treatment and/or therapeutic uses. Automated blood collection systems, however, use a specialized machine to separate the collected blood into the one or more components as it is collected from the donor. In certain variations, the automated blood collection systems may push back to the donor the unselected components of the one or more components.

Often, automated blood collection systems include prediction function software or programs that use information received and/or collected about the donor to determine appropriate flow rates and volumes that should result in final products that are within the limits of volumes, concentration, and ending cells counts for the individual donor as determined by government and/or blood center regulations and guidelines. Automated blood collection systems are also often configured to continuously monitor and tune and the collection process using the real time data to maintain accepted donation levels (usually referred to as the hypovolemic limit). For example, when one or more pumps (and in particular, the plasma pump(s)) are not appropriately loaded, and because of the positive pressure from the centrifuge(s), more volume can enter a collection bag than the particular automated blood collection system believes it has pump. In response to such situations, automated blood collection systems are often configured to monitor the draw and return volumes in a return reservoir and accumulates discrepancies overtime, applying the accumulated volume to the preselected levels (e.g., a hypovolemic limit) as set, for example, by governmental and/or blood center regulations and guidelines and alerting an alarm (and in certain instances, terminating the donation) to ensure that the failure mode does not lead to unsafe conditions for the donor. Using this configuration, however, false alarms can occur—that is, when the one or more pumps are examined, the pump(s) are actually properly loaded. Accordingly, it may be desirable to develop systems and methods of using the same that can more accurately monitor the real-time volumes.

### SUMMARY

This section provides a general summary of the disclosure and is not a comprehensive disclosure of its full scope or all of its features.

In at least one example embodiment, the present disclosure provides a method for using an automated blood collection system to collect blood components from a subject. The method may include separating plasma from whole blood as received from the subject; initiating an alert when a predicted accumulated volume loss is greater than a configured removal level, the predicted accumulated volume loss depending on an accumulated volume that includes the separated volume of plasma and the configured removal level being defined by a hypovolemic level or the pre-selected maximum for the subject; and in response to the alert and feedback from an operator of the automated blood collection system, ending the collection process, continuing the collection, or initiating platelet and red blood cell separation.

In at least one example embodiment, the method may further include determining the predicted accumulated volume loss. The predicted accumulated volume loss may be determined by summing a current predicted platelet volume less than a volume of anticoagulants predicted in the platelet, a current predicted plasma volume less than a volume of anticoagulants predicted in the plasma, a current predicted red blood cell volume less than a volume of anticoagulants predicted in the red blood cell production, and an accumulated volume detected by a reservoir monitor.

In at least one example embodiment, the method may further include determining the configured removal level by multiplying the hypovolemic level or the pre-selected maximum for the subject by a volume accuracy offset value.

In at least one example embodiment, the volume accuracy offset value may be greater than or equal to about 1.02 to less than or equal to about 1.1.

In at least one example embodiment, the volume accuracy offset value may be 1.06.

In at least one example embodiment, the ending of the collection may include initiating rinseback.

In at least one example embodiment, the continuing of the collection may include continuing to separate plasma from the whole blood as received from the subject.

In at least one example embodiment, during the plasma separation, the method may further include comparing a sum of an actual plasma volume collected and a current predicted platelet volume and a current predicted red blood cell volume to a total blood volume limit.

In at least one example embodiment, when the sum of the actual plasma volume collected and the current predicted platelet volume and the current predicted red blood cell volume is greater than the total blood volume limit, the method may further include initiating platelet and red blood cell separation.

In at least one example embodiment, when the sum of the actual plasma volume collected and the current predicted platelet volume and the and current predicted red blood cell volume is less than the total blood volume limit, the method may further include continuing to separate plasma from the whole blood as received from the subject.

In at least one example embodiment, the total blood volume limit may be 15% of a total blood volume as calculated for the individual subject.

In at least one example embodiment, the total blood volume limit may be multiplied by a volume accuracy offset value before being compared to the sum of the actual plasma volume collected and the current predicted platelet volume and the current predicted red blood cell volume.

In at least one example embodiment, the volume accuracy offset value may be greater than or equal to about 1.02 to less than or equal to about 1.1.

In at least one example embodiment, after the initiation of the platelet and red blood cell collection, the method may further include initiating rinseback.

In at least one example embodiment, the present disclosure provides a method for using an automated blood collection system to collect blood components from a subject. The method may include separating plasma from whole blood as received from the subject; comparing a predicted accumulated volume loss to a configured removal level, the predicted accumulated volume loss depending on an accumulated volume that includes the separated volume of plasma and the configured removal level that is defined by a hypovolemic level or the pre-selected maximum for the subject; and if the predicted accumulated volume loss is greater than the configured removal level, generating an alert that includes at least three prompts for a user of the automated blood collection system, where a first prompt of the at least three prompts may include ending the collection process in response to the alert, a second prompt of the at least three prompts may include continuing the separation of the plasma from the whole blood bypassing the alert, and a third prompt of the at least three prompts may include ending a plasma collection phase and initiating a platelet collection phase, a red blood cell collection phase, or a combination of the platelet collection phase and the red blood cell collection phase.

In at least one example embodiment, the method may further include determining the predicted accumulated volume loss determining the configured removal level. The predicted accumulated volume loss may be determined by summing a current predicted platelet volume less than a volume of anticoagulants predicted in the platelet, a current predicted plasma volume less than a volume of anticoagulants predicted in the plasma, a current predicted red blood cell volume less than a volume of anticoagulants predicted in the red blood cell production, and an accumulated volume detected by a reservoir monitor. The configured removal level may be determined by multiplying the hypovolemic level or the pre-selected maximum for the subject by a volume accuracy offset value.

In at least one example embodiment, when the alert is bypassed, the method may further include comparing a sum of an actual plasma volume collected and a current predicted platelet volume and a current predicted red blood cell volume to a total blood volume limit.

In at least one example embodiment, when the sum of the actual plasma volume collected and the current predicted platelet volume and the current predicted red blood cell volume is greater than the total blood volume limit, the platelet and red blood cell collection may be initiated, and when the sum of the actual plasma volume collected and the current predicted platelet volume and the current predicted red blood cell volume is less than the total blood volume limit, the method may further include continuing the separation of the plasma from the whole blood.

In at least one example embodiment, the total blood volume limit may be 15% of a total blood volume as calculated for the individual subject.

In at least one example embodiment, the total blood volume limit may be multiplied by a volume accuracy offset value before being compared to the sum of the actual plasma volume collected and the current predicted platelet volume and the current predicted red blood cell volume.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a flowchart illustrating an example method for monitoring and maintaining safe volume levels when using an automated blood collection system in accordance with at least one example embodiment of the present disclosure;

FIG. 2 is an example alert that may appear on a user interface of an automated blood collection system when a predicted accumulated volume loss is greater than or equal to the product of a volume accuracy offset value and a configured removal level in accordance with at least one example embodiment of the present disclosure; and

FIG. 3 is an example alert that may appear on a user interface of an automated blood collection system when a possible amount of collected plasma remains high and a plasma collection phase has been ended in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some example embodiments, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

The present disclosure relates to means for and methods of monitoring the collection of one or more blood components (like platelets, red blood cells, and plasma) using an automated blood collection system, like the blood collection systems described in U.S. Pat. No. 10,585,085, titled COLLECTING COMPONENTS OF A FLUID and issued Mar. 10, 2020 and/or U.S. Pat. No. 9,758,764, titled SEPARATING COMPOSITE LIQUIDS and issued Sep. 12, 2017 and/or U.S. Pat. No. 10,618,060, titled CENTRIFUGE SAFETY MECHANISM and issued April 14, 202 and/or U.S. Pat. No. 10,166,322, titled GAIN IN SEPARATION PROCESSES WITH CONTROL LOOP and issued Jan. 1, 2019 and/or U.S. Pat. No. 9,440,011, titled HYBRID BLOOD COMPONENT STORAGE BAG AND METHOD OF MAKING SUCH BAG and issued Sep. 13, 2016 and/or U.S. Pat. No. 8,523,750, titled METHOD AND APPARATUS FOR EXTRACTING PLATELETS WITH REDUCED PLASMA CARRYOVER and issued Sep. 3, 2013 and/or U.S. Pat. No. 8,123,713, titled SYSTEM AND METHODS FOR COLLECTING PLASMA PROTEIN FRACTIONS FROM SEPARATED BLOOD COMPONENTS and issued Feb. 28, 2012 and/or U.S. Pat. No. 7,780,618, titled EXTRACORPOREAL BLOOD PROCESSING APPARATUS AND METHODS WITH PRESSURE SENSING and issued Aug. 24, 2010, the entire disclosures of which are hereby incorporated by references. Methods of using automated blood collection systems often generally include collecting whole blood from a donor, separating the whole blood into one or more selected components (like platelets, red blood cells, and plasma), and pushing back other components not selected (like white blood cells) back to the donor.

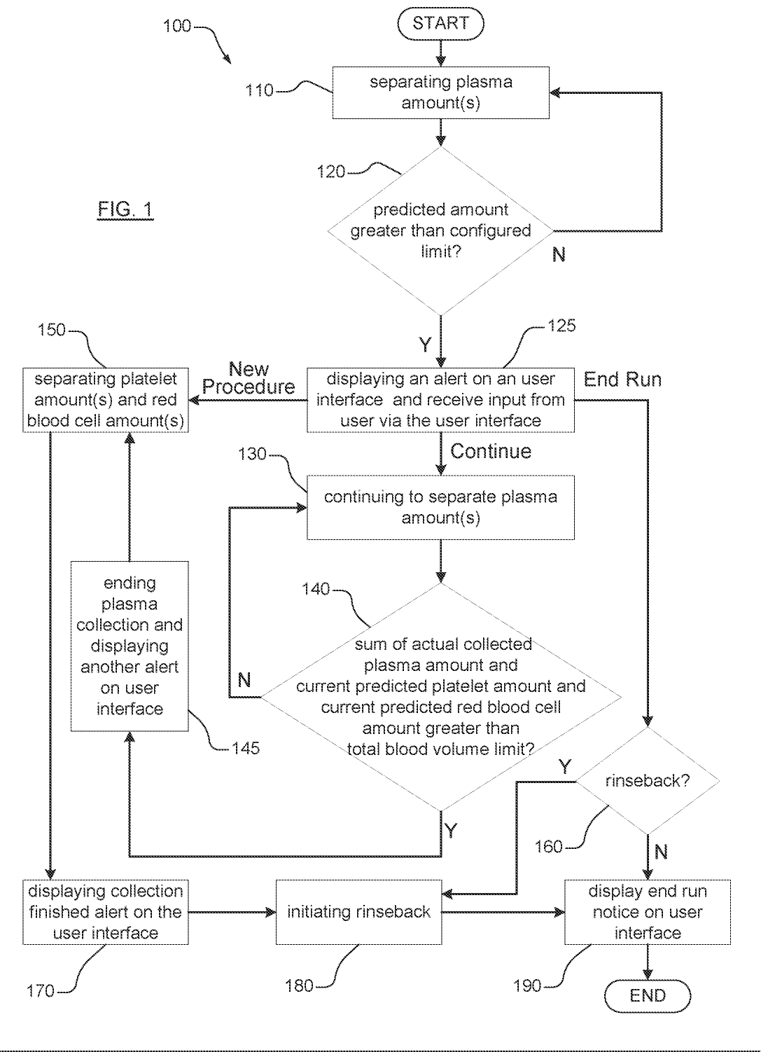

FIG. 1 illustrates an example method 100 for monitoring and maintaining collected plasma volumes or amounts within preselected levels (e.g., a hypovolemic limit) as set, for example, by governmental and/or blood center regulations and guidelines and referred to, for example, as the “configured removal level” when using an example automated blood collection system. The configured removal level is a donor-specific safe amounts or volumes of selected blood component(s) for collection from the donor or subject and may be determined using user or operator or donor inputs or stored information (such as, sex, height, weight, hematocrit, and/or platelet count) and also measured information from the ongoing collection (such as, inlet volume and/or blood volumes thus far collected).

The method 100 may include initiating a plasma collection draw cycle that includes completing one or more cycles or sequences or phases to separate or collected or removed or extracted 110 one or more amounts or volumes of plasma from whole blood collected or received from the donor. The plasma may be separated or collected or removed or extracted 110 from the whole blood using one or more centrifuge systems of the automated blood collection system. During the plasma separation or collection or removal or extraction, the method 100 may include determining 120 if a predicted accumulated volume loss or predicted accumulated amount loss for the donor or subject is greater than the configured removal level. In at least one example embodiment, the determining 120 may include monitoring both the amounts or volumes pumped into and out of a reservoir and ascertaining if the different between the two amounts or volumes is greater than a predetermined or normal threshold (e.g., about 7 milliliters). If the different is greater than the predetermined or normal threshold, than a potential failure of loading of the pump may be assumed and the method 100 may continue to method step 125. If however, the different is less than the predetermined or normal threshold, a potential failure of loading of the pump is not assumed and the method 100 may continue with the plasma separation or collection or removal or extraction (i.e., method step 110).

In at least one example embodiment, the configured removal level may be multiplied by a volume accuracy offset value before comparing the configured removal level to the predicted accumulated volume loss. The product of the configured removal level and the volume accuracy offset value may be referred to in certain instances as a corrected configured removal level and/or an adjusted configured removal level. The volume accuracy offset value may help to account for any volume offset. In at least one example embodiment, the volume accuracy offset value may be greater than or equal to about 1.02 to less than or equal to about 1.1, and in certain aspects, optionally 1.06. If the predicted accumulated volume loss is greater than or equal to the corrected configured removal level, the method 100 may continue to method step 125. If the predicted accumulated volume loss is less than the corrected configured removal level, the method 100 may continue with the plasma separation or collection or removal or extraction (i.e., method step 110).

Although not illustrated, it should be appreciated that, in at least one example embodiment, the method 100 may also include determining the predicted accumulated volume loss, which may also be referred to as the volume or amount to be removed. Calculating the predicted accumulated volume loss may be an iterative process that includes incorporating real-time data. For example, in at least on example embodiment, the predicted accumulated volume loss may be equal to the sum of a current predicted platelet volume less than a volume of anticoagulants (AC) predicted in the platelet, a current predicted plasma volume less than a volume of anticoagulants predicted in the plasma, a current predicted red blood cell volume less than a volume of anticoagulants predicted in the red blood cell production, and a volume accumulated towards a reservoir shutdown monitor. More simply, the predicted accumulated volume loss=(current predicted platelet volume−volume of anticoagulants predicted in the platelet)+(current predicted plasma volume−volume of anticoagulants predicted in plasma)+(current predicted red blood cell volume−volume of anticoagulants predicted in red blood cell product)+volume accumulated toward reservoir shutdown monitor.

Although not illustrated, it should be appreciated that, in at least one example embodiment, the method 100 may also include at least one of determining the current predicted platelet volume, determining the volume of anticoagulants predicted in the platelet, determining the current predicted plasma volume, determining the volume of anticoagulants predicted in plasma, determining the current predicted red blood cell volume, determining the volume of anticoagulants predicted in red blood cell product, and determining the volume accumulated toward reservoir shutdown monitor. In at least one example embodiment, the volume accumulated towards a reservoir shutdown monitor may be the difference between the amounts or volumes pumped into and out of a reservoir, as noted above.

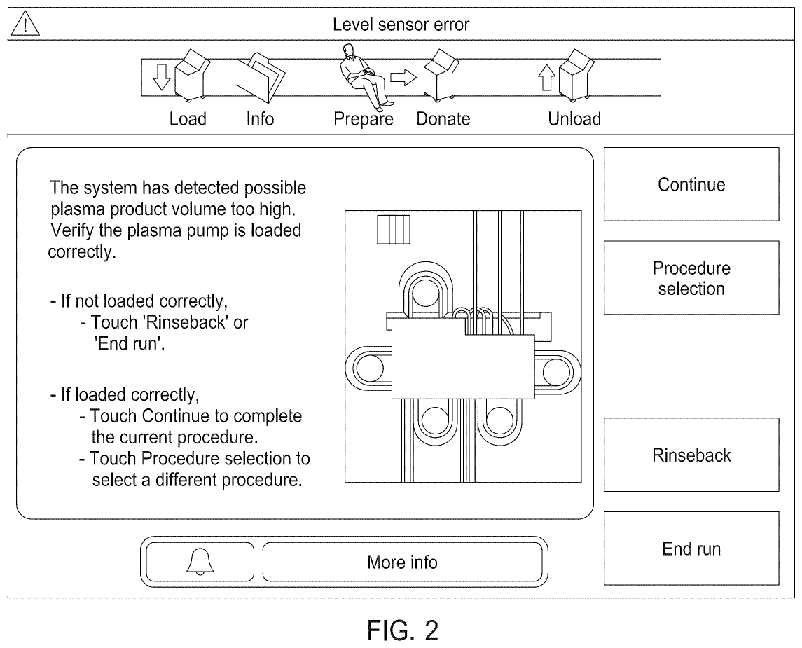

With renewed reference to FIG. 1, if the predicted accumulated volume loss is greater than or equal to the (corrected) configured removal level, the method 100 may include generating or sending or initiating an alert or alarm (such as illustrated in FIG. 2) to an user or graphical interface or screen of the automated blood collection system and/or stopping the one or more pumps of the automated blood collection system causing the plasma collection to cease. The user interface may also include a flag that the collected plasma may be contaminated, for example, with white blood cells. As illustrated in FIG. 2, the alert pushed to the user interface may include one or more prompts. For example, using the user interface, a user or operator of the blood collection system, after investigation, may choose to end or continue the collection or to enter a new patient procedure. For example, as illustrated in FIG. 1, if the user or operator selects to continue the collection, the method 100 may proceed to method step 130; if the user or operator enters a new procedure, the method 100 may proceed to method step 150; and if the user or operator chooses to end the run, the method 100 may proceed to method step 160.

Per the user's or operation's selection, the method 100 may include continuing the plasma separation or collection or removal or extraction at method step 130. During the ongoing or continued plasma separation or collection or removal or extraction, the method 100 may include determining 140 if a sum of actual collected plasma volume and the current predicted platelet volume and a current predicted red blood cell volume is greater than or equal to or in some instances near a total blood volume limit. In at least one example embodiment, the total blood volume limit may be a hard limit of 15% of a total blood volume as calculated for the individual donor.

In at least one example embodiment, before comparing the total blood volume limit with the sum of the actual collected plasma volume and the current predicted platelet volume and the current predicted red blood cell volume, the total blood volume limit may be multiplied by a volume accuracy offset value before comparing to the sum of the actual collected plasma volume and the current predicted platelet volume and to the current predicted red blood cell volume to account for any volume offset. The volume accuracy offset value used to adjust the total blood volume limit may be the same as or different from the volume accuracy offset value used to adjust the configured removal level. For example, in at least one example embodiment, the volume accuracy offset value to adjust the total blood volume limit may be greater than or equal to about 1.02 to less than or equal to about 1.1, and in certain aspects, optionally 1.06. The product of the total blood volume limit and the volume accuracy offset value may be referred to in certain instances as a corrected or adjusted total blood volume limit.

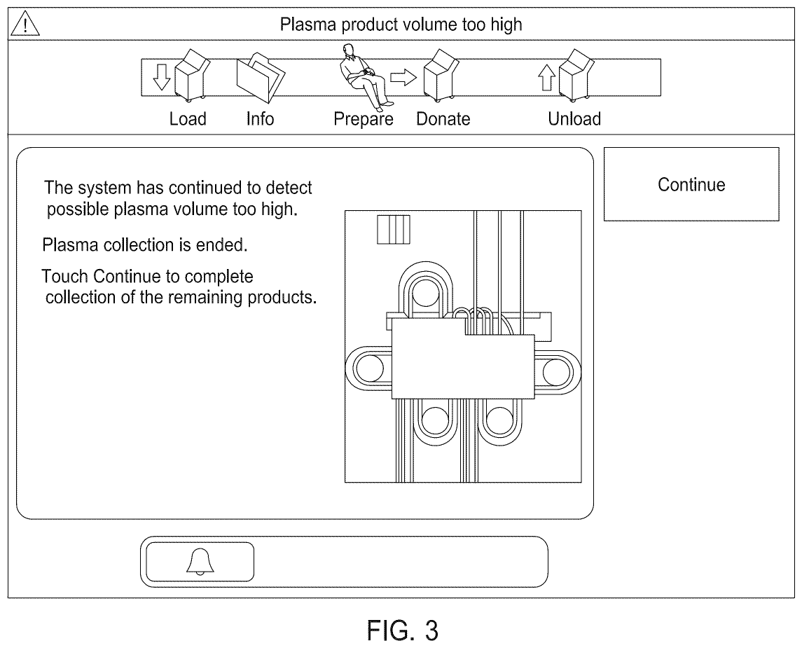

If the sum of the actual collected plasma volume and the current predicted platelet volume and the current predicted red blood cell volume is less than the (corrected) total blood volume limit, the method 100 may continue with the plasma separation or collection or removal or extraction (i.e., method step 130). If the sum of the actual collected plasma volume and the current predicted platelet volume and the current predicted red blood cell volume is greater than or equal to the (corrected) total blood volume limit, the method 100 may continue to method step 145, where the method 100 includes ending plasma collection and displaying another or second alert (for example, as illustrated in FIG. 3) regarding the plasma separation or collection or removal or extraction on the user interface of the automated blood collection system. The another alert, as illustrated in FIG. 3, may include a message that the system continues to detect that the plasma volume is possibly too high and that the plasma collection has been ended. The another alert may include a prompt directing the user or operator to select the prompt to initiate or continue collection of other blood products, like platelets and red blood cells. The user interface may also include a flag when displaying the another alert that the collected plasma may be contaminated, for example, with white blood cells.

With renewed reference to FIG. 1, the method 100 may include, either in response to the sum of the actual collected plasma volume and the current predicted platelet volume and the current predicted red blood cell volume being greater than or equal to the (corrected) total blood volume limit or in response to actions by the user or operator at method steps 120 of 145, completing one or more cycles or sequences or phases to separate or collect or remove or extract 150 one or more platelet volumes and/or one or more red blood cell volumes from whole blood collected or received from the donor. The amount(s) of platelets and/or amount(s) of red blood cells may be separated or collected or removed or extracted 150 from the whole blood as available after the removal of the one or more amounts of plasma using one or more centrifuge systems of the automated blood collection system. After the platelets and/or red blood cells are collected, the method 100 may include displaying 170 a collection finish screen on the user interface, after which rinseback may be initiated 180 during which blood components not collected and/or excessed material are returned or pushed back to the donor. Once rinseback occurs, the method 100 may include displaying 190 an end run notice on the user interface and ending the collection.

If the user or operator chooses to end the run when the initial alert is displayed, the method 100 may include a decision point 160 where the user or operator determines to initiate or bypass rinseback. If the user or operator decides to initiate rinseback, the method 100 may proceed to 180. If however, the user or operator decides to bypass rinseback, the method 100 may include displaying 190 an end run notice on the user interface and ending the collection.

The present configuration allows the collection to continue beyond the plasma alert and up to an upper limit preselected levels (e.g., a hypovolemic limit) as set, for example, by governmental and/or blood center regulations and guidelines, allows platelet and red blood cell collection to run to completion because the predictions are used for the plasma logic, provides the user or operator an option to select a lower volume procedure as needed, and uses the actual volume collected into a plasma collection bag of the automated blood collection system instead of the end of run predicted volume so as to enable the user or operator to collect, in most instances, usable volumes

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for using an automated blood collection system to collect blood components from a source, the method comprising:
separating a first blood component from whole blood as received from the source;
initiating an alert when a predicted accumulated volume loss is greater than a configured removal level, the predicted accumulated volume loss depending on an accumulated volume that includes the separated volume of the first blood component and the configured removal level being defined by a hypovolemic level or the pre-selected maximum for the source; and
in response to the alert, feedback from an operator of the automated blood collection system, or a combination thereof, ending the collection process, continuing the collection, or initiating separation of a second blood component, a third blood component, or both a second blood component and a third blood component.

2. The method of claim 1, wherein the method further includes determining the predicted accumulated volume loss by summing a current predicted platelet volume less a volume of anticoagulants predicted in the platelet, a current predicted plasma volume less a volume of anticoagulants predicted in the plasma, a current predicted red blood cell volume less a volume of anticoagulants predicted in the red blood cell production, and an accumulated volume detected by a reservoir monitor.

3. The method of claim 1, wherein the method further includes determining the configured removal level by multiplying the hypovolemic level or the pre-selected maximum for the source by a volume accuracy offset value.

4. The method of claim 3, wherein the volume accuracy offset value is greater than or equal to about 1.02 to less than or equal to about 1.1.

5. The method of claim 4, wherein the volume accuracy offset value is 1.06.

6. The method of claim 1, wherein the ending the collection process includes initiating rinseback.

7. The method of claim 1, wherein the continuing of the collection includes continuing to separate the first blood component from the whole blood as received from the source.

8. The method of claim 7, wherein during the first blood component separation, the method further includes comparing a sum of an actual first blood component volume collected and a current predicted second blood component volume and a current predicted third blood component volume to a total blood volume limit.

9. The method of claim 8, wherein when the sum of the actual first blood component volume collected and the current predicted second blood component volume and the current predicted third blood component volume is greater than the total blood volume limit, the method further includes initiating separation of the second blood component.

10. The method of claim 8, wherein when the sum of the actual first blood component volume collected and the current predicted second blood component volume and the current predicted third blood component volume is less than the total blood volume limit, the method further includes continuing to separate the first blood component from the whole blood as received from the source.

11. The method of claim 8, wherein the total blood volume limit is 15% of a total blood volume as calculated for the specific source.

12. The method of claim 8, wherein the total blood volume limit is multiplied by a volume accuracy offset value before being compared to the sum of the actual first blood component volume collected and the current predicted second blood component volume and the current predicted third blood component volume.

13. The method of claim 12, wherein the volume accuracy offset value is greater than or equal to about 1.02 to less than or equal to about 1.1.

14. The method of claim 1, wherein the first blood component includes plasma, the second blood component includes platelets, and the third blood component includes red blood cells.

15. A method for using an automated blood collection system to collect blood components from a source, the method comprising:
receiving whole blood from the source;
separating a first component from the whole blood;
comparing a predicted accumulated volume loss to a configured removal level, the predicted accumulated volume loss depending on an accumulated volume that includes the separated volume of the first component and the configured removal level being defined by a hypovolemic level or the pre-selected maximum for the source; and
if the predicted accumulated volume loss is greater than the configured removal level, generating an alert that includes at least three prompts for a user of the automated blood collection system, a first prompt of the at least three prompts including ending the collection process in response to the alert, a second prompt of the at least three prompts including continuing the separation of the plasma first component from the whole blood bypassing the alert, and a third prompt of the at least three prompts including ending a first component collection phase and initiating a second component collection phase, a third component collection phase, or a combination of the second component collection phase and the third component collection phase.

16. The method of claim 15, wherein the method further includes:
determining the predicted accumulated volume loss by summing a current predicted platelet volume less a volume of anticoagulants predicted in the platelet, a current predicted plasma volume less a volume of anticoagulants predicted in the plasma, a current predicted red blood cell volume less a volume of anticoagulants predicted in the red blood cell production, and an accumulated volume detected by a reservoir monitor; and
determining the configured removal level by multiplying the hypovolemic level or the pre-selected maximum for the source by a volume accuracy offset value.

17. The method of claim 15, wherein when the alert is bypassed, the method further includes comparing a sum of an actual first component volume collected, a current predicted second component volume, and a current predicted third component volume to a total blood volume limit.

18. The method of claim 17, wherein when the sum of the actual first component volume collected and the current predicted second component volume and the current predicted third component volume is greater than the total blood volume limit, the second component and third component collection is initiated, and when the sum of the actual first component volume collected and the current predicted second component volume and the current predicted third component volume is less than the total blood volume limit, the method further includes continuing the separation of the first component from the whole blood.

19. The method of claim 17, wherein the total blood volume limit is 15% of a total blood volume as calculated for the individual source.

20. The method of claim 17, wherein the total blood volume limit is multiplied by a volume accuracy offset value before being compared to the sum of the actual first component volume collected and the current predicted second component volume and the current predicted third component volume.
